(19) United States
(12) Patent Application Publication
(10) Pub. No.: US 2009/0105521 A1
(43) Pub. Date: Apr. 23, 2009

(54) Systems and methods for assessing and treating medical conditions related to the central nervous system and for enhancing cognitive functions

(76) Inventor: Jonathan Bentwich, Zipori (IL)

Correspondence Address: DICKSTEIN SHAPIRO LLP, 1825 EYE STREET NW, Washington, DC, 20006-5403, US

(21) Appl. No.: 12/285,417

(22) Filed: Oct. 3, 2008

Related U.S. Application Data
(63) Continuation-in-part of application No. 12/153,037, filed on May 13, 2008.
Application No. 12/153,037 is a continuation of application No. 10/904,505, filed on Nov. 14, 2004, now abandoned.
(60) Provisional application No. 60/960,575, filed on Oct. 4, 2007.
(60) Provisional application No. 60/522,286, filed on Sep. 13, 2004.

(51) Int. Cl. A61N 2/02; A61N 1/36; A61N 5/06

(52) U.S. Cl. 600/9; 607/45; 607/88

(57) ABSTRACT

Systems and methods for diagnosing and treating various brain-related conditions, and/or for modifying at least one of cognitive, behavioral, or affective functions or skills in individuals. The method of diagnosing and treating a brain-related condition includes the steps of: (i) identifying at least a brain region associated with the brain-related condition; (ii) stimulating the brain region by employing at least one electrical, magnetic, electromagnetic, and photoelectric stimulus; (iii) optionally, stimulating at least one cognitive feature associated with the brain region; and (iv) optionally, subjecting the brain region to a treatment including at least one of cell replacement therapy, cell regenerative therapy and cell growth.

### CROSS-REFERENCE TO RELATED APPLICATIONS

This application claims the benefit of U.S. Provisional Application No. 60/960,575, filed Oct. 4, 2007, entitled “System and Method for Assessment and Treatment of Central Nervous System Medical Implications and Indications,” the entire disclosure of which is incorporated by reference in its entirety herein. This application is also a continuation-in-part of U.S. application Ser. No. 12/153,037, filed May 13, 2008, which is a continuation of U.S. application Ser. No. 10/904,505, filed Nov. 14, 2004, which in turn claims the benefit of U.S. Provisional Application No. 60/522,286, filed Sep. 13, 2004, the entire disclosures of which are also incorporated by reference in their entirety herein.

This application is related to Attorney Docket No. N2222.0007/P007, entitled “Systems and Methods for Treatment of Medical Conditions related to the Central Nervous System and for Enhancing Cognitive Functions,” filed on even day herewith, and incorporated by reference in its entirety herein, which non-provisional application claims the benefit of U.S. Provisional Application No. 60/960,574, filed Oct. 4, 2007, entitled “Method and System for Enhancement of Cognitive Functions and Helmet for Treatment of Central Nervous System Medical Implications,” the entire disclosure of which is incorporated by reference in its entirety.

### FIELD OF THE INVENTION

The present invention relates to systems and methods for assessing and treating medical conditions and cognitive impairments associated with the neural system.

### BRIEF SUMMARY OF THE INVENTION

The present invention provides systems and methods for assessing and treating various medical conditions associated with the neural system. The present invention also provides systems and methods for assessing and treating various cognitive conditions in individuals.

Other features and advantages of the present invention will become apparent from the following description of the invention, which refers to the accompanying drawings.

### BRIEF DESCRIPTION OF THE DRAWINGS

FIG. 1 is a schematic block-diagram of an integrative neuro-cognitive system according to an exemplary embodiment of the present invention;

FIG. 2 is a schematic block-diagram of the NEURODIAGNOSTICS MODULE of the system of FIG. 1;

FIG. 3 is a schematic block-diagram of the REGIONS OF INTEREST COMPUTATIONAL MODULE of the system of FIG. 1;

FIG. 4 is a schematic block-diagram of the BRAIN TRAIT COMPUTATION MODULE of the system of FIG. 1;

FIG. 5 is a schematic block-diagram of the TREATMENT MODULE of the system of FIG. 1; and

FIG. 6 is a schematic block-diagram of the STIMULATION MODULE of the system of FIG. 1.

### DETAILED DESCRIPTION OF THE INVENTION

The examples provided below detail the various embodiments of the present invention. Other features, embodiments, and advantages of the invention beyond those discussed in the detailed description will become more apparent to those skilled in the art in views of details provided herein. Those skilled in the art should appreciate that many changes may be made to the present invention without departing from the scope or spirit of the present invention.

The present invention provides systems and methods for assessing and treating various medical conditions associated with the neural system. The present invention also provides systems and methods for assessing and treating various cognitive conditions in individuals.

The present invention provides systems and apparatus configured to identify and treat various brain-related conditions and/or to assess and modify (for example, enhance) at least one of cognitive, behavioral, or affective function or skill in individuals. The system may include at least one stimulator. A suitable stimulator includes, but is not limited to, a first stimulator, which may include at least one of invasive and non-invasive brain stimulation devices, and a second stimulator which is operatively connected to the first stimulator. The first stimulator is configured to identify at least one brain region associated with a brain-related condition and to stimulate the identified brain region by employing at least one of electrical, magnetic, electromagnetic, and photoelectric stimuli. The second stimulator is configured to modify at least one cognitive function associated with the identified brain region. The first and second stimulators may form a single integrated device or, alternatively, may form separate parts of the device. The first and second stimulators are configured to operate simultaneously or sequentially.

The present invention also provides methods of diagnosing and treating various brain-related conditions and/or of modifying at least one cognitive, behavioral, or affective function or skill in individuals. The method of diagnosing and treating a brain-related condition or for enhancing a cognitive function may include the steps of: (i) identifying at least a brain region associated with the brain-related condition or the cognitive function; (ii) stimulating the brain region by employing a stimulus such as electrical, magnetic, electromagnetic, and photoelectric stimuli; (iii) optionally, stimulating at least one cognitive feature associated with the brain region of at least step (i); (iv) optionally, subjecting the brain region of at least step (i) to a treatment involving at least one of cell replacement therapy, cell regenerative therapy and cell growth; and (v) optionally, subjecting the brain region of at least step (i) to a pharmacological treatment.

The present invention provides integrative neuro-cognitive systems for diagnosing and treating various brain-related diseases, and/or for assessing and enhancing particular cognitive, behavioral, or affective functions (or skills) in brain-related cognitive functions in normal individuals (based on an individual-based comparison of structural or functional or cognitive functioning with corresponding statistical health or brain diseases norms or with statistical norms for cognitively enhanced functions). The integrative neuro-cognitive system of the present invention also provides subsequent neuronal electrical or electromagnetic stimulation, and convergent cognitive stimulation of the identified diseased brain regions in an individual or sub-enhanced cognitive function or functions of brain regions.

The invention also provides neurodiagnostic computational systems and methodology for differentially diagnosing an individual with a particular brain-related disease or diseases, along with a specification of the individual's particular functional, structural, or cognitive abnormalities. Alternatively, the invention provides neurodiagnostic computational systems and methodology for identifying those particular cognitive function or functions, which may be further enhanced in an individual relative to cognitively enhanced standards for brain functions. Additionally, the invention also provides apparatus and methods of computing a precise individual-based brain stimulation, and corresponding cognitive stimulation parameters, needed to stimulate the identified disease-related brain loci, or to enhance an identified cognitive skill or function.

The invention further provides apparatus, systems and methods for stimulating the relevant brain regions and corresponding cognitive functions, while continuously monitoring and adjusting the brain and cognitive stimulation parameters for a given individual or a disease or a particular cognitive enhancement function, based on a comparison of pre- and post-stimulation neurodiagnostic measurements of the relevant brain function, structure, and corresponding cognitive functions.

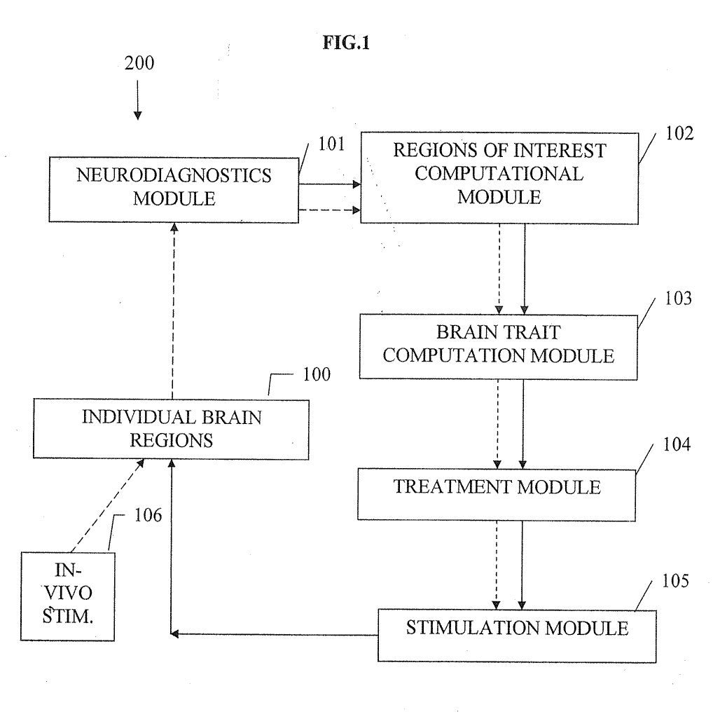

Referring now to the drawings, where like elements are designated by like reference numerals, FIGS. 1-6 illustrate various structural elements of system 200 of the present invention configured to diagnose and treat medical conditions associated with the neural system, and/or to enhance cognitive functions in mammals.

Reference is made to FIG. 1, which illustrates INDIVIDUAL BRAIN REGIONS 100 that are pathological functional or structural brain features, or cognitive performance features in an individual, which are associated with a specific brain-related disease that is identified by a NEURODIAGNOSTICS MODULE 101 (FIG. 1). NEURODIAGNOSTICS MODULE 101 measures the functional activation or structural maps, or corresponding cognitive performance in an individual for a particular task (or tasks) or during a resting period. NEURODIAGNOSTICS MODULE 101 transfers this information to REGIONS OF INTEREST COMPUTATIONAL MODULE 102, which identifies those particular brain regions in an individual whose structure, function, or cognitive functions are deviant from their corresponding statistically-established health norms, or from their corresponding statistical norms for cognitively enhanced performance in a particular task.

REGIONS OF INTEREST COMPUTATIONAL MODULE 102 outputs these identified statistically-deviant or cognitively-enhanced brain regions in a given individual for analysis in a BRAIN TRAIT COMPUTATION MODULE 103. The BRAIN TRAIT COMPUTATION MODULE 103 determines whether or not any of these identified brain regions statistically fits within known structural, functional, or cognitive pathophysiology of a particular brain-related disease. Alternatively, BRAIN TRAIT COMPUTATION MODULE 103 determines whether or not any of these identified brain regions statistically fits within established norms for enhanced or excellent cognitive or behavioral performance (in a particular task or skill or skills). Thus, for example, in the case of Autism Spectrum Disorder (ASD), statistically-established norms indicate that autistic children or individuals exhibit an abnormal deficient activation (as well as structurally decreased size) of the left hemisphere's (LH) typical Broca's and Wernicke's language regions, while abnormally hyperactivating (or structurally enlarged) contralateral (RH) Broca's and Wernicke's regions. Therefore, in cases in which the REGIONS OF INTEREST COMPUTATIONAL MODULE 102 identifies such abnormal hypoactivation of the LH's Broca's and Wernicke's language regions (with or without an accompanying hyperactivation of the contralateral RH's Broca's and Wernicke's regions), the COMPUTATIONAL MODULE 102 then outputs these regions to the BRAIN TRAIT COMPUTATION MODULE 103, to determine whether or not any of these identified brain regions statistically fits within known structural, functional, or cognitive pathophysiology of Autism Spectrum Disorder (ASD).

Alternatively, in the case of Alzheimer's disease (or any other memory loss that is due to aging, dementia or mild cognitive impairment (MCI)), statistically established norms indicate that such memory impairment is associated with decreased structure and function of the hippocampus and other medial temporal structures, as well as decreased connectivity between frontal and posterior brain regions and facial recognition regions, or structural, functional, or cognitive impairment of the cerebellum (associated with impaired motor coordination and semantic memory or verbal capability loss), or impairment of mood and executive functioning regions (such as the left prefrontal region and cingulate gyrus and frontal lobe). Therefore, in cases in which the REGIONS OF INTEREST COMPUTATIONAL MODULE 102 identifies such abnormally-decreased structural or functional values of these brain structures, these brain regions are output to the BRAIN TRAIT COMPUTATION MODULE 103, to determine whether or not any of these identified brain regions statistically fits within known structural, functional, or cognitive pathophysiology of Alzheimer's, MCI, dementia, or age-related memory loss, or other aging illnesses. In those cases in which the identified regions of interest or cognitive performance levels match the brain disease, or match the neural functional, structural, or cognitive levels of a sub-cognitively enhanced performance in a particular task or tasks, the TREATMENT MODULE 104 computes the precise individual-based brain and cognitive stimulation parameters needed to stimulate the identified INDIVIDUAL BRAIN REGIONS 100 that are necessary to improve the functional, structural or cognitive disease indices, or to enhance performance in a particular task or tasks.

The STIMULATION MODULE 105 receives input from the TREATMENT MODULE 104 regarding an individual-based brain and cognitive stimulation including their integrated neuro-cognitive stimulation parameters. Additionally and/or optionally, an IN-VIVO STIMULATOR 109 may be combined with the STIMULATION MODULE 105. In an exemplary embodiment, IN-VIVO STIMULATOR 109 may include in-vivo transplantation or regenerative or stem-cell insertion of neuronal cells or tissue or supportive cells targeting the same INDIVIDUAL BRAIN REGIONS 100.

A feedback may be also combined with the STIMULATION MODULE 105, and following the STIMULATION MODULE 105. The feedback may include a post-stimulation measurement carried out by the NEURODIAGNOSTICS MODULE 101 which then undergoes all the sequential computational steps including: the REGIONS OF INTEREST COMPUTATIONAL MODULE 102, the BRAIN TRAIT COMPUTATION MODULE 103, the TREATMENT MODULE 104, and the STIMULATION MODULE 105. All feedback computational steps are aimed towards monitoring and adjusting the individual-based brain and corresponding cognitive stimulation parameters continuously, based on the potential improvement in functional, structural, or corresponding cognitive stimulation in an individual following the administration of brain stimulation and corresponding cognitive stimulation (e.g., until a certain pathophysiological disease threshold has been transcended indicating clinical improvement in that individual or, alternatively, until a certain cognitive enhancement threshold has been transcended indicating an enhancement of a particular cognitive function or functions in an individual).

Each of the components of FIG. 1 (i.e., the NEURODIAGNOSTICS MODULE 101, the REGIONS OF INTEREST COMPUTATIONAL MODULE 102, the BRAIN TRAIT COMPUTATION MODULE 103, the TREATMENT MODULE 104, and the STIMULATION MODULE 105) can function independently or separately, or in any possible combination with each other.

In accordance with one embodiment of the present invention, the NEURODIAGNOSTICS MODULE 101 is configured to translate functional or structural neuroimaging data into statistically valid individual functional activation patterns and statistically valid individual structural maps. The NEURODIAGNOSTICS MODULE 101 is also configured to compare individual cognitive performance data with statistically established health norms.

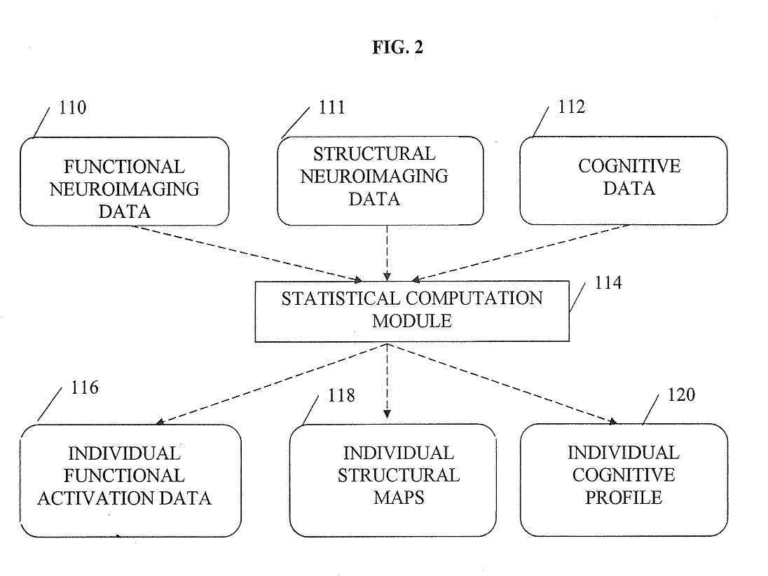

Reference is now made to FIG. 2, which illustrates a simplified block diagram of the NEURODIAGNOSTICS MODULE 101 of system 200 of FIG. 1. NEURODIAGNOSTICS MODULE 101 is configured to obtain a FUNCTIONAL NEUROIMAGING DATA 110, a STRUCTURAL NEUROIMAGING DATA 111, and a COGNITIVE DATA 112, that are then fed into a STATISTICAL COMPUTATION MODULE 114. As shown in FIG. 2, STATISTICAL COMPUTATION MODULE 114 is configured to compute an INDIVIDUAL FUNCTIONAL ACTIVATION DATA 116, an INDIVIDUAL STRUCTURAL MAPS 118, and an INDIVIDUAL COGNITIVE PROFILE 120.

The FUNCTIONAL NEUROIMAGING DATA 110 includes various neuroimaging measurements of activation across different brain regions of a specific individual, during the performance of a particular cognitive or behavioral task. Another possible measurement of the FUNCTIONAL NEUROIMAGING DATA 110 includes neuroimaging measurements of a specific individual while at rest. This data can be obtained through the use of various magnetic resonance imagining (MRI), functional magnetic resonance imagining (fMRI), positron emission tomography (PET), single photon emission computerized tomography (SPECT), electroencephalography (EEG) and event related potentials (ERP), among many others.

The STRUCTURAL NEUROIMAGING DATA 110 includes various neuroimaging measurements of an individual's brain structure. A non-limiting example of structural mapping is the MRI (although, as detailed above, other devices such as PET and SPECT are also capable of generating structural images).

The COGNITIVE DATA 112 includes measurements of cognitive performance of an individual in a wide range of possible cognitive or behavioral tests, which may include but are not limited to: response times, accuracy, measures of attention, memory, learning, executive function, language, intelligence, personality measures, mood, and self-esteem, among others. The cognitive data may be obtained through computerized, paper and pencil, interviewing, performance tests or other forms of administering the cognitive or behavioral tests. The cognitive data may be obtained via verbal, written, visual or tactile responses which are input into the computer in various forms.

As shown in FIG. 2, the FUNCTIONAL NEUROIMAGING DATA 110, the STRUCTURAL IMAGING DATA 111, and the COGNITIVE DATA 112 are input into the STATISTICAL COMPUTATION MODULE 114 which compares each of these types of data to statistically established norms, to determine an INDIVIDUAL FUNCTIONAL ACTIVATION DATA 116, an INDIVIDUAL STRUCTURAL MAPS 118, and an INDIVIDUAL COGNITIVE PROFILE 120. Various computational softwares for performing those computational and analyses are available, such as ICA, SPM and AutoROI, among many others.

Based on the analysis of the STATISTICAL COMPUTATION MODULE 114 of the individual's functional patterns relative to the statistically established norms, the INDIVIDUAL FUNCTIONAL ACTIVATION DATA 116 provides unique brain activation patterns of an individual performing a specific cognitive or behavioral task, or while resting, relative to a statistically established norm.

Similarly, based on the analysis of the STATISTICAL COMPUTATION MODULE 114 of the individual's structural brain images relative to statistically established norms, the INDIVIDUAL STRUCTURAL MAPS 118 provides unique brain structure of an individual.

Based on the analysis of the STATISTICAL COMPUTATION MODULE 114 of the individual's cognitive performance relative to statistically established norms, the INDIVIDUAL COGNITIVE PROFILE 120 includes that individual's unique cognitive capabilities, skills or functions.

The NEURODIAGNOSTICS MODULE 101 may consist of the FUNCTIONAL NEUROIMAGING DATA 110, the STRUCTURAL NEUROIMAGING DATA 111, the COGNITIVE DATA 112, together or separately, or in any combination. However, the STATISTICAL COMPUTATION MODULE 114 is a part of the NEURODIAGNOSTICS MODULE 101 in any combination.

A constraint imposed on the possible combinations of these components is that, if the FUNCTIONAL NEUROIMAGING DATA 110 inherently exists in the individual, then the INDIVIDUAL FUNCTIONAL ACTIVATION DATA 116 must exist; if the STRUCTURAL NEUROIMAGING DATA 111 inherently exists in the individual, then the INDIVIDUAL STRUCTURAL MAPS 118 must exist; and, if the COGNITIVE DATA 112 inherently exists in the individual, then the INDIVIDUAL COGNITIVE PROFILE 120 must exist.

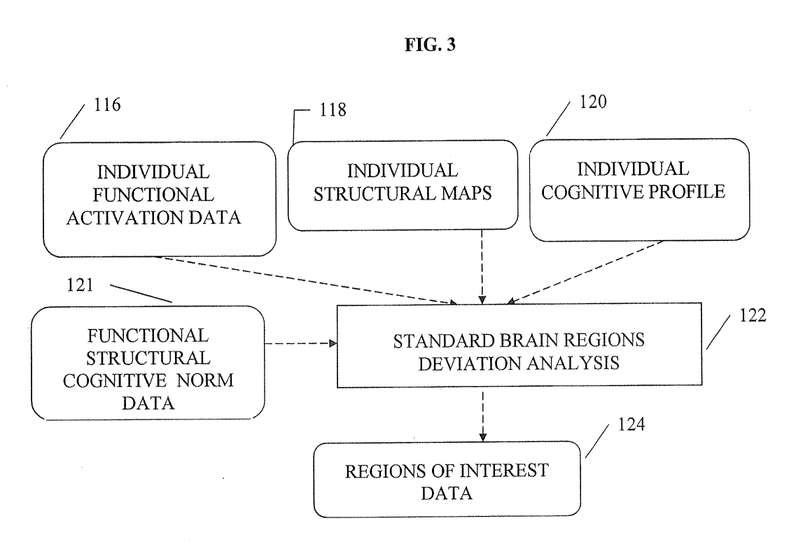

Reference is now made to FIG. 3, which is a simplified illustration of the REGIONS OF INTEREST COMPUTATION MODULE 102 of system 200 of FIG. 1. The REGIONS OF INTEREST COMPUTATION MODULE 102 is configured to identify a disease-specific and individual-specific pathophysiological brain regions. Alternatively, the REGIONS OF INTEREST COMPUTATION MODULE 102 is configured to identify the particular functional or structural brain loci, or corresponding cognitive characteristics, that are different in a given normal individual from their corresponding attributes in statistical standard of excellence or enhanced performance in a particular cognitive skill or function associated with a particular brain region.

Input from the INDIVIDUAL FUNCTIONAL ACTIVATION DATA 116, the INDIVIDUAL STRUCTURAL MAPS 118, and the INDIVIDUAL COGNITIVE PROFILE 120 of FIG. 2, and a FUNCTIONAL, STRUCTURAL, COGNITIVE NORM DATA 121 are received by the STANDARD BRAIN REGIONS DEVIATION ANALYSIS 122, which determines which brain regions exhibit a deviation from statistically established health norms in terms of functional activation patterns, structure, or corresponding cognitive performance levels and is output as the REGIONS OF INTEREST DATA 124. Alternatively, the STANDARD BRAIN REGIONS DEVIATION ANALYSIS 122 is configured to determine which brain regions exhibit a deviation from a statistical established norm for functional activation patterns, brain structure, and cognitive features of a particular excellent or enhanced cognitive or behavioral performance that is output as the REGIONS OF INTEREST DATA 124.

Each of the three INDIVIDUAL FUNCTIONAL ACTIVATION DATA 116, INDIVIDUAL STRUCTURAL MAPS 118, and INDIVIDUAL COGNITIVE PROFILE 120 can function independently or separately, or in any possible combination with the other two modules. However, at least one of these three modules must accompany the FUNCTIONAL, STRUCTURAL, COGNITIVE NORM DATA 121 and the STANDARD BRAIN REGIONS DEVIATION ANALYSIS 122, to compute and output the REGIONS OF INTEREST DATA 124 (which are the particular functional, structural, or corresponding cognitive brain regions which exhibit statistically deviant values relative to the distribution of the normal population or, alternatively, relative to the distribution of enhanced cognitive performance corresponding to functional, structural, or cognitive performance levels).

In accordance with one embodiment of the present invention, the STANDARD BRAIN REGIONS DEVIATION ANALYSIS 122 relies on statistical computation which compares an individual's functional activation patterns to statistically established health norms (which may rely on known standards of normal brain activation during the performance of a particular cognitive or behavioral task or tasks or at rest, or it may rely on a statistical comparison of the individual to a sufficiently large sample of functional activation patterns in a group of normal matched controls performing a particular cognitive-behavioral task or tasks). The comparison of the individual's functional activation patterns, brain structure, or cognitive performance to statistically established health norms relies on a statistical contrast between the individual's cognitive performance values (pixel by pixel, or region by region, functional and structural, or particular brain regions) with the corresponding values of a normally distributed healthy control group or population.

The goal of any one of a variety of statistical procedures known in the art is to determine the likelihood of the individual's functional, structural or cognitive values (parsed by cell, region, brain structure, lobe or hemisphere levels) as belonging to the normal distribution of corresponding functional, structural, or cognitive values in normal controls. Different confidence intervals, significance thresholds, and means of reducing error rate etc. can be utilized to determine those regions of interest which are different in the individual relative to the control group.

In accordance with another embodiment of the present invention, the STANDARD BRAIN REGIONS DEVIATION ANALYSIS 122 may rely on statistical computation which compares an individual's functional activation patterns to statistically established norms for excellent or enhanced particular cognitive, or behavioral performance, in above-average individuals, or following enhancing brain stimulation of the regions corresponding to a particular cognitive function, or enhancing cognitive training of the same particular cognitive function or skill. The comparison of the individual's functional activation patterns, brain structure or cognitive performance to statistically-established norms of functional, structural, or cognitive performance in individuals who exhibit excellent cognitive performance in a particular task or skill can rely on a statistical contrast of the individual's pixel by pixel, or region by region, functional and structural or cognitive performance values with the corresponding values of a normally-distributed healthy control group or population. The goal of any one of a variety of statistical procedures known in the art is to determine the likelihood of the individual's functional, structural, or cognitive values (parsed by cell, region, brain structure, lobe or hemisphere levels) as belonging to the (normal) distribution of corresponding functional, structural, or cognitive values in excellent or enhanced cognitive performance in a particular task or skill for individual normal controls, or following a cognitive training of that particular function, or through enhancing that cognitive function through stimulation of the corresponding brain regions.

The STANDARD BRAIN REGIONS DEVIATION ANALYSIS 122 outputs the REGIONS OF INTEREST DATA 124, the particular structural brain loci, functional brain regions, and cognitive features that are deviant from the statistically established functional or structural brain norms. Alternatively, the STANDARD BRAIN REGIONS DEVIATION ANALYSIS 122 outputs the REGIONS OF INTEREST DATA 124 that may includes the particular structural brain loci, functional brain regions, and cognitive features that are different from the statistically established functional or structural brain norms for a standard of a particular excellent or enhanced cognitive performance.

Several examples for possible REGIONS OF INTEREST DATA 124 in the case of an individual at risk for developing (or already exhibiting) abnormal functional, structural or corresponding cognitive performance abnormalities associated with Alzheimer disease are as follows: abnormally deficient activation of left frontal, left prefrontal, Broca's, Wernicke's, hippocampus and related regions, anterior cingulated, and also motor, medial temporal gyrus, anthreonal gyrus, cerebellum, and a decline in functional connectivity measures between some or all of these regions. Structural abnormalities may also consist of a decrease in these structures volume or connecting fibers between these neuronal regions. In the case of autism spectrum disorder (ASD), structural abnormalities are evidenced by reversed functional activation of right hemisphere RH instead of left hemisphere LH language regions activation patterns in ASD children (and adults) relative to normal matched controls, e.g., hypoactivation of LH's Broca's, Wernicke's regions but hyperactivation of these contralateral regions in the RH in the ASD relative to matched controls. For “Theory of Mind” social cognition ASD deficits, functional hypoactivation of the Amygdala, fusiform gyrus, and dysfunction of inter-hemispheric connectivity measures may occur. Additionally, a generalized RH dysfunction in the ASD individuals relative to controls which may manifest as a generalized RH hyperactivation in Theory of Mind paradigms, at resting conditions or in language paradigms, may occur.

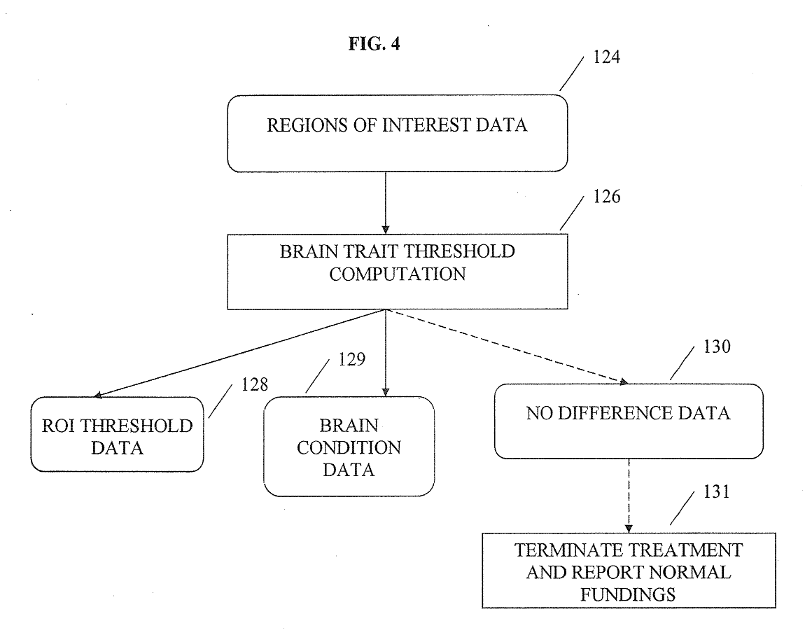

Reference is now made to FIG. 4, which depicts the BRAIN TRAIT COMPUTATION MODULE 103 of system 200 of FIG. 1. BRAIN TRAIT COMPUTATION MODULE 103 is configured to determine whether or not the identified REGIONS OF INTEREST DATA 124 signify a likelihood of the individual being afflicted by a specific functional, structural, or corresponding cognitive impairment related to a specific brain-related disease. Alternatively, the BRAIN TRAIT COMPUTATION MODULE 103 of FIG. 1 is configured to determine whether or not the identified REGIONS OF INTEREST DATA 124 signify the likelihood of an individual being below enhanced or excellent functional, structural, or corresponding cognitive-task performance criteria (e.g., in terms of functional, structural, or cognitive values relative to their corresponding values in a sample of individuals with excelled performance).

The REGIONS OF INTEREST DATA 124 (which are those brain regions for which the functional activation, structure, or corresponding cognitive performance has been determined to be statically different in an individual than in the control group or, alternatively, relative to a sample of cognitively enhanced performance) is input into the BRAIN TRAIT THRESHOLD COMPUTATION 126. The BRAIN TRAIT THRESHOLD COMPUTATION 126 determines which of these REGIONS OF INTEREST DATA 124 has a functional activation, or structural properties, or corresponding cognitive performance values that are different from disease-specific statistical threshold values that have a high predictive value for the existence of a specific disease in an individual at the time of testing or prospectively at different time points. Alternatively, the REGIONS OF INTEREST DATA 124 is input into the BRAIN TRAIT THRESHOLD COMPUTATION 126 which determines whether these REGIONS OF INTEREST DATA 124 have functional activation or structural values that are the same as, or different from, the statistically determined functional or structural values threshold for a particularly enhanced cognitive function or functions.

In cases in which the BRAIN TRAIT THRESHOLD COMPUTATION 126 determines that the REGIONS OF INTEREST (ROI) DATA 124 are same as, or exceed, the threshold for functional or structural values of a particular region or regions that have been determined as characterizing a particular disease, then it will output an ROI THRESHOLD DATA 128 and a BRAIN CONDITION DATA 129. For those functional, structural, or corresponding cognitive performance threshold values of a particular brain-related disease which are characterized as being below the statistically computed values of the normal control population, then, if an individual's REGIONS OF INTEREST DATA 124 are below the above-mentioned disease-specific threshold, the BRAIN TRAIT THRESHOLD COMPUTATION 126 will output the ROI THRESHOLD DATA 128 as consisting of all the REGIONS OF INTEREST DATA 124 that are below-threshold regions for a particular brain-related disease specified by the BRAIN CONDITION DATA 129. In those cases in which the BRAIN TRAIT THRESHOLD COMPUTATION 126 detects statistically significant functional, structural, or corresponding cognitive performance values in an individual that exceed the disease-specific threshold values or, alternatively, are below the disease-specific threshold in cases in which the functional, structural, or corresponding cognitive performance values have been determined to be statistically below those of normal controls, the BRAIN TRAIT THRESHOLD COMPUTATION 126 will also output a BRAIN CONDITION DATA 129 with a specification of what particular brain-related disease is statistically reliably associated with these above-threshold (or below-threshold as explained above) functional, structural, or corresponding cognitive performance values in a given individual.

In cases in which the functional, structural, or corresponding cognitive performance values in an individual have not exceeded the disease-specific threshold (or in cases in which the disease-specific threshold is below the statistical values in the normal population and the individual's ROI THRESHOLD DATA 128 is above these disease-specific thresholds), then the BRAIN TRAIT THRESHOLD COMPUTATION 126 will output a NO DIFFERENCE DATA 130 (e.g., indicating that no functional, structural, or cognitive patterns exist in the individual that are different from the statistical distribution of normal individuals). In this case, the NO DIFFERENCE DATA 130 instigates a TERMINATE TREATMENT AND REPORT NORMAL FINDINGS 131, which terminates the diagnostic phase of the invention with an output to the individual, or the treating clinician, that the individual is not likely to suffer from any brain-related disease and, therefore, no treatment is warranted.

In cases in which the BRAIN TRAIT THRESHOLD COMPUTATION 126 determines that the REGIONS OF INTEREST DATA 124 are same as, or exceed, the threshold for functional or structural values of a particular region or regions that have been determined as characterizing an enhanced performance or function in a particular cognitive task or skill, then it will output an ROI THRESHOLD DATA 128 and a BRAIN CONDITION DATA 129. For those functional or structural values that are associated with a particularly enhanced cognitive skill or function which are characterized as being below the statistically computed values of the normal control population, then, if an individual's REGIONS OF INTEREST DATA 124 are below the above-mentioned cognitive enhanced threshold, the BRAIN TRAIT THRESHOLD COMPUTATION 126 will output the ROI THRESHOLD DATA 128 consisting of all the REGIONS OF INTEREST DATA 124 that are below-threshold regions. In those cases in which the BRAIN TRAIT THRESHOLD COMPUTATION 126 detects statistically significant functional or structural values in an individual that exceed the cognitively enhanced threshold values or, alternatively, are below the cognitive enhanced threshold values in cases in which the functional or structural values have been determined to be statistically below those of normal controls, the BRAIN TRAIT THRESHOLD COMPUTATION 126 also outputs a BRAIN CONDITION DATA 129 which includes a specification of what particular cognitively enhanced skills or functions are statistically reliably associated with these above-threshold (or below-threshold as explained above) functional, structural, or corresponding cognitive performance values in a given individual.

In those cases in which the BRAIN TRAIT THRESHOLD COMPUTATION 126 outputs the ROI THRESHOLD DATA 128 and BRAIN CONDITION DATA 129, the ROI THRESHOLD DATA 128 includes the identification of all the pixels, or cellular, or regional, or hemispheric brain regions for which the functional, structural, or corresponding cognitive performance levels in an individual have been computed to exceed the disease-specific threshold in an individual or be below the disease-specific threshold (as shown above), and an indication of the precise functional or structural or cognitive values of each of these pixels, or cellular or regional or hemispheric loci relative to their corresponding disease-specific threshold. In those cases in which the BRAIN TRAIT THRESHOLD COMPUTATION 126 outputs the ROI THRESHOLD DATA 128 and BRAIN CONDITION DATA 129, and in which the ROI THRESHOLD DATA 128 includes the identification of all pixels, or cellular, or regional, or hemispheric brain regions for which the functional, structural, or corresponding cognitive performance levels in an individual have been computed to be lower than the enhanced cognitive performance level in a particular cognitive task or function (or be below the particularly enhanced cognitive threshold as shown above), the ROI THRESHOLD DATA 128 also specifies the precise functional, structural, or cognitive values at each of the identified pixels, cellular or regional or hemispheric loci—along with their corresponding statistically computed thresholds.

In cases in which the functional, structural, or corresponding cognitive performance values in an individual have not exceeded the disease-specific threshold (or in cases in which the disease-specific threshold is below the statistical values in the normal population and the individual's ROI THRESHOLD DATA 128 is above these disease-specific thresholds), then the BRAIN TRAIT THRESHOLD COMPUTATION 126 outputs a NO DIFFERENCE DATA 130 (e.g., indicating that no functional, structural, or cognitive patterns exist in the individual that are different from the statistical distribution of normal individuals). In this case, the NO DIFFERENCE DATA 130 instigates a TERMINATE TREATMENT AND REPORT NORMAL FINDINGS 131, which terminates the diagnostic phase of the invention with an output to the individual or the treating clinician that the individual is not likely to suffer from any brain-related disease and, therefore, that no treatment is warranted.

In cases in which the functional or structural values in an individual have not exceeded the cognitively-enhanced threshold (or in cases in which the cognitively enhanced threshold is below the statistical values in the normal population and the individual's ROI THRESHOLD DATA 128 is above these particular cognitively enhanced threshold), then the BRAIN TRAIT THRESHOLD COMPUTATION 126 outputs a NO DIFFERENCE DATA 130 (e.g., indicating that no functional, structural, or cognitive patterns exist in the individual that are different from the statistical distribution of cognitively enhanced functional or structural features). In this case, the NO DIFFERENCE DATA 130 instigates a TERMINATE TREATMENT AND REPORT NORMAL FINDINGS 131, which terminates the diagnostic phase of the invention with an output to the individual or the treating clinician that the individual is not likely to benefit from any cognitive enhancement treatment.

The computation carried out by the BRAIN TRAIT THRESHOLD COMPUTATION 126 is based upon a statistical comparison of an individual's functional activation, brain structure, or cognitive performance with a statistical distribution of the corresponding functional, structural, or cognitive performance in particular brain-related diseases. Alternatively, the computation carried out by the BRAIN TRAIT THRESHOLD COMPUTATION 126 may be based upon a statistical comparison of an individual's functional activation, brain structure, or cognitive performance with a statistical distribution of the corresponding functional, structural, or cognitive performance for particularly enhanced cognitive skills or functions. These statistical comparisons consist of a pixel by pixel, cellular, regional, or hemispheric comparison of that individual's REGIONS OF INTEREST DATA 124 with its corresponding statistical norms for specific diseases or, alternatively, for particularly enhanced cognitive functions. These statistical norms for normal functional, structural, or corresponding cognitive performance may be obtained through meta-analysis (or other statistical procedures) for averaging scientifically published data quantifying functional, structural, or corresponding cognitive performance levels at different pixel, cellular, regional or hemispheric levels, and across different neuroimaging paradigms in a specific disease and a particular sub-phenotype or stage of the specific disease.

Alternatively, these statistically computed norms for normal brain functioning, structure, and corresponding cognitive performance may be obtained through a sufficiently large sample of normal vs. diseased individuals for a specific disease, with subsequent statistical methods being utilized to normalize the distribution of normal controls vs. diseased individuals which would result in the computation of a specific statistical threshold for each pixel, cell, region or hemisphere—above or below which values in an individual are likely to represent a specific disease, sub-phenotype or stage of a particular disease. Moreover, varying the significance level, confidence interval, power of test, effect size or other statistical measures which quantify the difference between a particular brain diseased population and normal control population based on a sample from these populations—may allow one to obtain different statistical (predictive) thresholds for distinguishing a brain-related disease from normal control values.

The BRAIN TRAIT THRESHOLD COMPUTATION 126 determination of the statistical threshold above- or below-which functional, structural, or corresponding cognitive performance levels are likely to represent a particular brain disease, sub-phenotype, or disease-stage depends upon the analysis of the normal vs. diseased sample distribution (i.e., in those cases in which the statistical analysis has demonstrated that the normal sample yields statistically reliable higher functional or structural values for a particular pixel, cell, region, or hemisphere than the disease sample, then the BRAIN TRAIT THRESHOLD COMPUTATION 126 will determine that values in an individual for that particular pixel, cell, region hemisphere etc. which are below the computed threshold for normal population values will be marked as a diseased region for a particular disease). Thus for example, statistical analyses have demonstrated that the normal sample yields statistically reliable higher functional or structural values for the LH's Broca's and Wernicke's regions than values for an autism sample. Therefore, the BRAIN TRAIT THRESHOLD COMPUTATION 126 will determine that an individual who exhibits functional activation, structural volume, or cognitive values for those particular brain regions which are below the computed threshold for the corresponding normal population values will be marked as a diseased region for autism, in that particular individual. Similarly, statistical analyses have demonstrated that the normal sample yields statistically reliable higher functional activation, structural volume, or cognitive values for the hippocampus, medial temoral structures, connectivity between frontal and posterior or facial recognition or cerebellum or cingulated values than for an Alzheimer's or MCI or demented or aging sample. Therefore, the BRAIN TRAIT THRESHOLD COMPUTATION 126 will determine that an individual who exhibits functional, structural, or cognitive values for those particular brain regions which are below the computed threshold for the corresponding normal population values will be marked as a diseased region for Alzheimer's or MCI or aging diseases.

Conversely, in those cases in which the statistical analysis has demonstrated that the normal sample yields statistically reliable lower functional or structural values for a particular pixel, cell, region, or hemisphere than the disease sample, then the BRAIN TRAIT THRESHOLD COMPUTATION 126 will determine that values in an individual for that particular pixel, cell, region hemisphere etc. which are above the computed threshold for normal population values will be marked as a diseased region for a particular disease. Thus, for example, statistical analyses have shown that the normal sample yields statistically reliable lower functional activation, or structural volume values for the RH's contralateral Broca's or Wernicke's regions than in a sample of autistic children. Therefore, the BRAIN TRAIT THRESHOLD COMPUTATION 126 will determine that values in an individual for the RH's contralateral Broca's or Wernicke's regions that are above the corresponding computed threshold for normal population values will be marked as a diseased region for autism spectrum disorder.

Similarly, in order for the BRAIN TRAIT THRESHOLD COMPUTATION 126 to compute the threshold for functional, structural, or corresponding values indicative of an enhanced cognitive performance in an individual at a particular task or tasks, a statistical comparison of normal vs. enhanced samples or populations will be performed for pixel by pixel, cellular, regional or hemispheric functional, structural or corresponding cognitive measures. In those cases in which the statistical analysis has demonstrated that the enhanced sample yields statistically reliable higher functional or structural values for a particular pixel, cell, region, or hemisphere than in the normal sample or population, the BRAIN TRAIT THRESHOLD COMPUTATION 126 will determine that values in an individual for that particular pixel, cell, region hemisphere etc. which are below the computed threshold for the enhanced population or sample will de determined as indicating that these cellular, regional, or hemispheric regions are indicative of sub-enhanced functional, structural, or corresponding cognitive performance levels in that particular individual. As such, an excitatory stimulation of these identified sub-enhanced brain regions in an individual may enhance their corresponding cognitive performance.

Conversely, in those cases in which the statistical analysis has demonstrated that the enhanced sample yields statistically reliable lower functional or structural values for a particular pixel, cell, region, or hemisphere than the normal sample or population, then the BRAIN TRAIT THRESHOLD COMPUTATION 126 will determine that values that are above the enhanced sample or population's threshold in an individual may indicate a sub-enhanced functional, structural, or corresponding cognitive level in an individual for a particular cognitive trait, performance or skill. As such, inhibitory stimulation of these identified sub-enhanced brain regions in an individual may enhance their corresponding cognitive performance.

The BRAIN TRAIT THRESHOLD COMPUTATION 126 determines whether or not the functional, structural, or corresponding cognitive performance levels in an individual are statistically the “same” or “different” in a given individual relative to their corresponding values in a normal population. Once the BRAIN TRAIT THRESHOLD COMPUTATION 126 has determined that particular REGIONS OF INTEREST DATA 124 do exceed the disease-specific statistical threshold or, alternatively, are below a particular enhanced performance threshold, then it outputs the BRAIN TRAIT DATA 127, which indicates which brain regions are abnormal functionally, structurally, or in terms of their association with particularly impaired cognitive performance, or alternatively which brain regions may be stimulated neuronally or cognitively to enhance a particular cognitive function or skill.

The BRAIN TRAIT THRESHOLD COMPUTATION 126 also outputs the BRAIN THRESHOLD DATA 128, which includes a pixel by pixel, cellular, brain region, or hemispheric values and cognitive performance thresholds for normal brain functioning or, alternatively, for enhanced brain functioning along with various statistical indices associated with these computational thresholds such as significance level, confidence intervals etc., or any other statistical measure that assesses the statistical difference between the REGIONS OF INTEREST DATA 124 functional, structural, or cognitive values and the statistically-established threshold for normal brain functioning. If, on the other hand, the BRAIN TRAIT THRESHOLD COMPUTATION determines that all of the REGIONS OF INTEREST DATA 124 do not exceed the disease-specific statistical threshold or, alternatively, are not below the particular enhanced cognitive performance threshold, then BRAIN TRAIT THRESHOLD COMPUTATION 126 outputs a NO DIFFERENCE DATA 129, which then leads to a TERMINATE TREATMENT AND REPORT NORMAL FINDINGS 130 (which terminates the operation of the medical device and notifies the patient or clinician that the individual is normal with no apparent brain-related disease or, alternatively, performs excellent a particular cognitive task and, therefore, cannot benefit from brain and cognitive stimulation geared towards enhancing particular cognitive skills).

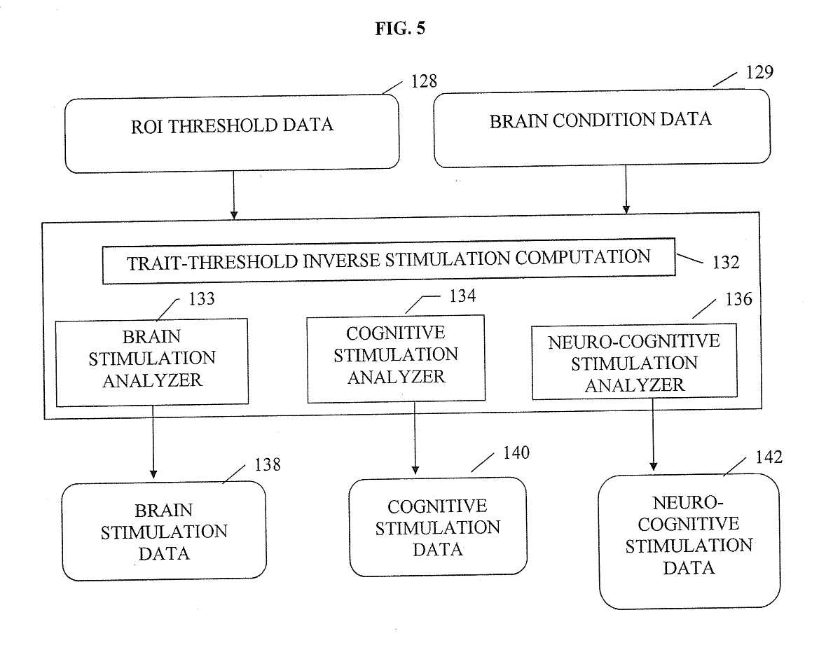

Reference is now made to FIG. 5, which illustrates the TREATMENT MODULE 104 of the system 200 of FIG. 1. The TREATMENT MODULE 104 is configured to determine the precise brain stimulation, cognitive stimulation, and neuro-cognitive stimulation parameters for an individual with a specific brain-related disease. Alternatively, the TREATMENT MODULE 104 is capable of determining the precise brain stimulation, cognitive stimulation and neuro-cognitive stimulation parameters for a normal individual to enhance a particular cognitive function.

The TREATMENT MODULE 104 includes the ROI THRESHOLD DATA 128 and the BRAIN CONDITION DATA 129 of FIG. 4, which are input into a TRAIT-THRESHOLD INVERSE STIMULATION COMPUTATION 132 that includes a BRAIN STIMULATION ANALYZER 133, a COGNITIVE STIMULATION ANALYZER 134, and a NEURO-COGNITIVE STIMULATION ANALYZER 136, which in turn produce a corresponding BRAIN STIMULATION DATA 138, a COGNITIVE STIMULATION DATA 140, and a NEURO-COGNITIVE STIMULATION DATA 140.

The TRAIT-THRESHOLD INVERSE STIMULATION COMPUTATION 132 is configured to compare between the ROI THRESHOLD DATA 128 functional, structural, or cognitive performance levels that are above or below disease-specific thresholds, or are above or below enhanced cognitive performance levels in an individual and their corresponding functional, structural, or corresponding cognitive performance thresholds, and the BRAIN CONDITION DATA 129, to determine the optimal brain, cognitive, or neuro-cognitive stimulation parameters.

A key computational principle guiding the function of the TRAIT-THRESHOLD INVERSE STIMULATION COMPUTATION 132 is that, to improve the functional, structural, or corresponding cognitive performance level in an individual suffering from a particular brain-related disease or, alternatively, to enhance the functional, structural, or corresponding cognitive performance level in a normal individual, it is necessary to stimulate the particularly identified ROI THRESHOLD DATA 128 regions in the inverse excitatory or inhibitory stimulation direction relative to the below or above threshold levels in a given individual. In this manner, in those cases in which an individual's functional, structural, or corresponding cognitive performance levels are below the threshold for corresponding normal functional, structural, or cognitive performance, then the TRAIT-THRESHOLD INVERSE STIMULATION COMPUTATION 132 will compute a generally excitatory brain or cognitive stimulation. For example, in those cases in which an individual's functional, structural, or corresponding cognitive performance levels have been characterized as belonging to autism spectrum disorder's hypoactivation (or abnormally small structure volume) of the LH's Broca's and Wernicke's language regions or of the Aygdala or fusiform gyrus which are below the threshold for corresponding normal functional, structural, or cognitive performance, then the TRAIT-THRESHOLD INVERSE STIMULATION COMPUTATION 132 will compute a generally excitatory brain or cognitive stimulation of these brain regions. Likewise, in those cases in which an individual's functional, structural, or corresponding cognitive performance levels have been characterized as belonging to Alzheimer's, aging, dementia, or MCI which is detected through a hypoactivation (or abnormally small structure volume) of the hippocampus, medial-temporal structures, impairment in connectivity between frontal and posterior or facial recognition regions, or cerebellum or cingulate function or structure, then the TRAIT-THRESHOLD INVERSE STIMULATION COMPUTATION 132 will compute a generally excitatory brain or cognitive stimulation of these brain regions.

Conversely, in those cases in which an individual's functional, structural or corresponding cognitive performance levels are above the threshold for corresponding normal functional, structural, or cognitive performance levels, then the TRAIT-THRESHOLD INVERSE STIMULATION COMPUTATION 132 will compute a generally inhibitory brain or cognitive stimulation. For example, in those cases in which an individual's functional, structural, or corresponding cognitive performance levels have been characterized as belonging to autism spectrum disorder characterized by a hypoactivation (or abnormally small structure volume) of the RH's contralateral Broca's and Wernicke's regions, then the TRAIT-THRESHOLD INVERSE STIMULATION COMPUTATION 132 will compute a generally excitatory brain or cognitive stimulation of these brain regions.

The same trait-threshold inverse stimulation principle also applies to the TRAIT-THRESHOLD INVERSE STIMULATION COMPUTATION 132 for cognitive enhancement. Specifically, in those cases in which an individual's functional, structural or corresponding cognitive performance levels are below the enhanced-cognitive performance threshold, then the TRAIT-THRESHOLD INVERSE STIMULATION COMPUTATION 132 will compute a generally excitatory brain or cognitive stimulation. Conversely, in those cases in which an individual's functional, structural or corresponding cognitive performance levels are above the cognitive-enhancement threshold, then the TRAIT-THRESHOLD INVERSE STIMULATION COMPUTATION 132 will compute a generally inhibitory brain or cognitive stimulation.

Specifically, the BRAIN STIMULATION ANALYZER 133 compares between ROI THRESHOLD DATA 128 functional levels that are above or below disease-specific thresholds, or are above or below particular cognitive enhancement thresholds, in a given individual and their corresponding functional threshold, while taking into consideration the BRAIN CONDITION DATA 129 particular brain-related disease, or the particular cognitive enhancement goal—to determine the optimal brain stimulation parameters in a given individual. For example, in cases in which an individual's functional or structural activation parameters are below the normal threshold in certain ROI THRESHOLD DATA 128 regions, then the BRAIN STIMULATION ANALYZER 133 will output excitatory brain stimulation parameters. Conversely, in cases in which an individual's functional or structural activation parameters are above the normal threshold in certain ROI THRESHOLD DATA 128 regions, then the BRAIN STIMULATION ANALYZER 133 will output inhibitory BRAIN STIMULATION DATA 138 parameters.

Similarly, the COGNITIVE STIMULATION ANALYZER 134 compares between ROI THRESHOLD DATA 128 cognitive levels that are above or below disease-specific thresholds, or are above or below particular cognitive enhancement thresholds, in a given individual and their corresponding cognitive thresholds, while taking into consideration the BRAIN CONDITION DATA 129 particular brain-related disease or diseases, or the particular cognitive enhancement goal—to determine the optimal cognitive stimulation parameters in a given individual. For example, in cases in which an individual's cognitive performance level is below the normal threshold for a particular task or function, then the COGNITIVE STIMULATION ANALYZER 133 will output an excitatory cognitive stimulation parameters. Conversely, in cases in which an individual's cognitive performance levels in a particular cognitive function are above the normal threshold, then the COGNITIVE STIMULATION ANALYZER 133 will output inhibitory COGNITIVE STIMULATION DATA 142 parameters (i.e., cognitive stimulation paradigm or training methodology which attempts to inhibit the abnormal (or sub-enhanced) cognitive function either directly or through the training or stimulation of its opposite or complimentary or other cognitive function, which in effect suppresses or diminishes the particular abnormal or sub-enhanced cognitive function).

Likewise, the NEURO-COGNITIVE STIMULATION ANALYZER 136 compares between ROI THRESHOLD DATA 128 functional, structural, or corresponding cognitive performance levels that are above or below disease-specific thresholds, or are above or below particular cognitive enhancement thresholds in a given individual and their corresponding functional threshold, while taking into consideration the BRAIN CONDITION DATA 129 of a particular brain-related disease, or the particular cognitive enhancement goal—in order to determine the optimal brain stimulation parameters in a given individual. However, in the case of the NEURO-COGNITIVE STIMULATION ANALYZER 136, the computation is geared towards identifying the optimal neuro-cognitive stimulation parameters (e.g., in terms of the correspondence between stimulating a specific brain region (or regions) in an excitatory or inhibitory manner and its corresponding cognitive stimulation of the same brain region (or regions) in an inhibitory or excitatory manner, the temporal overlap or separation between the neuronal brain stimulation, and cognitive stimulation of the same or different brain regions, etc.). Thus, based on the ROI THRESHOLD DATA 128 indication of which particular brain region (or regions) is above or below the disease specific or cognitively-enhanced threshold, and which BRAIN CONDITION DATA 129 disease does such above or below threshold individual levels belong to, the NEURO-COGNITIVE STIMULATION ANALYZER 136 computes the above-mentioned optimal neuro-cognitive stimulation parameters.

The specific intensity, duration, loci, interval, and other parameters of brain stimulation computed by the BRAIN STIMULATION ANALYZER 133 are determined based on the input from the BRAIN CONDITION DATA 129 in conjunction with the above-mentioned trait-threshold inverse stimulation principle (e.g., in cases in which the individual's ROI THRESHOLD DATA 128 functional or structural levels are relatively far from the BRAIN CONDITION DATA 129 disease threshold or cognitive enhancement threshold, then the inhibitory or excitatory stimulation parameters would tend to be of higher intensity, duration, multiple brain loci etc., and vice versa).

An exemplary embodiment of the present invention encompasses the TREATMENT MODULE 104's tentative ROI THRESHOLD DATA 128 of particular brain-related diseases such as Alzheimer's and ASD's BRAIN CONDITION DATA 129. Specifically, in the case of Alzheimer's, the ROI THRESHOLD DATA 128 is expected to include any one of these regions or any combination thereof: abnormally deficient activation of left frontal, left prefrontal, Broca's, Wernicke's, hippocampus and related regions, anterior cingulated, and also motor, medial temporal gyrus, anthreonal gyrus, cerebellum, and a decline in functional connectivity measures between some or all of these regions. Structural abnormalities may also exist as a decrease in these structures' volume or connecting fibers between these neuronal regions.

In the case of autism spectrum disorder (ASD), ROI THRESHOLD DATA 128 is expected to include any one of these regions or any combination thereof: reversed functional activation of right hemisphere RH instead of left hemisphere LH language regions activation patterns in ASD children (and adults) relative to normal matched controls (e.g., hypoactivation of LH's Broca's, Wernicke's regions but hyperactivation of these contralateral regions in the RH in the ASD relative to matched controls). For “Theory of Mind” social cognition ASD deficits, functional hypoactivation of the Amygdala, fusiform gyrus, and dysfunction of inter-hemispheric connectivity measures may occur. Additionally, a generalized RH dysfunction in the ASD individuals relative to controls which may manifest as a generalized RH hyperactivation in Theory of Mind paradigms, at resting conditions or in language paradigms, may occur.

Accordingly, an exemplary and only illustrative embodiment of the system of the present invention includes BRAIN STIMULATION DATA 138, or COGNITIVE STIMULATION DATA 142, or NEURO-COGNITIVE STIMULATION DATA 140 excitatory stimulation of the left frontal or left prefrontal or Broca's or Wernicke's or hippocampus and related regions or anterior cingulate or motor or medial temporal gyrus, or anthreonal gyrus or cerebellum, or the functional connectivity between some or all of these regions or stimulation of any combination of these regions—in the case of Alzheimer's disease. Likewise, milder cases of Mild Cognitive Impairment (or any other form of age-related memory loss or dementia) may call for similar stimulation of some or all of these brain regions. In the case of ASD, an exemplary embodiment of the system of the present invention may include BRAIN STIMULATION DATA 138, or COGNITIVE STIMULATION DATA 140, or NEURO-COGNITIVE STIMULATION DATA 140 excitatory stimulation of any one of these regions or any combination thereof: Broca's or Wernicke's regions, or Amygdala or fusiform gyrus or of inter-hemispheric connections. Additionally, ASD may call for the BRAIN STIMULATION DATA 138, or COGNITIVE STIMULATION DATA 140, or NEURO-COGNITIVE STIMULATION DATA 140 inhibitory stimulation of the contralateral Broca's or Wernicke's RH regions or a generalized inhibitory stimulation of the RH.

To enhance various cognitive functions or skills, the corresponding brain regions should be stimulated excitatorily, i.e., hippocampus or temporal lobe or cingulated gyrus for memory or learning enhancement, frontal or prefrontal cortex for executive functions, concentration, learning, intelligence; motor cortex or cerebellum for motor functions and coordination, visual cortex for enhancing visual functions, inhibitive amygdale for fear and anxiety reduction with or without left frontal and prefrontal excitatory stimulation; Enhancement of self-esteem or mood or well-being-excitatory stimulation of left prefrontal or frontal, or inhibitive stimulation of the right prefrontal gyrus. In all these cases, corresponding cognitive stimulation may be applied (e.g., stimulus which improves or enhances the disease brain-related or cognitive function or enhances the desired cognitive function or functions).

An important aspect of the TRAIT-THRESHOLD STIMULATION COMPUTATION 132 is the principle of disease-specific or cognitive enhancement specific neuroplasticity computation, which underlies the computation carried out by the NEURO-COGNITIVE STIMULATION ANALYZER 136. This principle embodies the adaptation of various neuro-cognitive stimulation parameters to a specific brain disease, or particular cognitive enhancement protocol, based on the identification of the specific neuroplasticity features that are associated with these particular brain disease or diseases, and cognitive enhancement protocol or protocols. Thus, the NEURO-COGNITIVE STIMULATION ANALYZER 136 takes into account the specific BRAIN CONDITION DATA 129 brain disease or cognitive enhancement goal in a particular individual and, based on this information in conjunction with known neuroplasticity information regarding these ROI THRESHOLD DATA 128 and BRAIN CONDITION DATA 129, the ROI THRESHOLD DATA 128 determines the optimal NEURO-COGNITIVE STIMULATION DATA 140.

The neuroplasticity stimulation parameters may include, for example, the following: the intensity of the brain and corresponding cognitive stimulation, their duration, onset and termination times, temporal overlap or separation, order and combination of all possible brain stimulation loci and their corresponding cognitive stimulations, among others. These parameters may all be dynamically changed or adjusted based on the post-stimulation NEURODIAGNOSTICS MODULE 100 and REGIONS OF INTEREST COMPUTATIONAL MODULE 102 and BRAIN TRAIT COMPUTATION MODULE 103 and TREATMENT MODULE 105.

One example of such NEURO-COGNITIVE STIMULATION ANALYZER 136 is the computation of the optimal neuroplasticity stimulation for treating Alzheimer's memory loss or other MCI, dementia, memory loss diseases, or memory enhancement diseases, which may include: excitatory 10-20 Hz TMS stimulation of the hippocampus or other temporal lobe regions or frontal or prefrontal regions or cingulate gyrus in any possible combination, which will be synchronized with memory enhancement or encoding or retrieval or recall or recognition or mnemonic or perceptual or auditory or semantic memory enhancement cognitive training or stimulation methodologies, to obtain the optimal neuroplasticity potential changes related to memory improvement (e.g., based on the computation of the best neuroplasticity parameters that allow for the most learning, encoding memory retrieval or formation pertaining to these particular ROI THRESHOLD DATA 128 and BRAIN CONDITION DATA 129).

The determination by the NEURO-COGNITIVE STIMULATION ANALYZER 136 of the optimal neuroplasticity parameters specific for a particular ROI THRESHOLD DATA 128 and BRAIN CONDITION DATA 129 may be derived from prior art findings regarding any particular combination of ROI THRESHOLD DATA 128 and BRAIN CONDITION DATA 129. Alternatively, it can be computed based on the present invention's post-stimulation dynamic feedback loop with the above-mentioned NEURODIAGNOSTICS MODULE 100, REGIONS OF INTEREST COMPUTATIONAL MODULE 102, BRAIN TRAIT COMPUTATION MODULE 103, TREATMENT MODULE 105 and STIMULATION MODULE 105. The latter feedback loop computation can allow computation of the most effective learning curve or NEURO-COGNITIVE STIMULATION DATA 140 for a particular ROI THRESHOLD DATA 128 and BRAIN CONDITION DATA 129 combination, either as monitored and adjusted dynamically in a given individual, or through a statistical meta-analysis or other statistical methodology for analyzing the effectiveness of various neuro-cognitive stimulation parameters for a particular ROI THRESHOLD DATA 128 and BRAIN CONDITION DATA 129 across multiple individuals having the same ROI THRESHOLD DATA 128 and BRAIN CONDITION DATA 129 combination. In this manner, the NEURO-COGNITIVE STIMULATION ANALYZER 136 (when embedded and integrated within the post-stimulation feedback loop mentioned above) offers an automatic learning potential for optimizing the neuro-cognitive stimulation parameters for any given ROI THRESHOLD DATA 128 and BRAIN CONDITION DATA 129 combination.

An important aspect of the present invention is the capacity of the BRAIN TRAIT COMPUTATION MODULE 103 to offer a differential diagnostic statistical tool for screening, evaluating, and diagnosing the existence of a particular brain-related disease in an individual at the time of testing, or to offer a reliable predictive diagnostic tool based on statistically reliable deviation of the REGIONS OF INTEREST 124 from the corresponding functional, structural, or cognitive performance distribution in the normal population or sample. In this manner, the BRAIN TRAIT COMPUTATIONAL MODULE 103 may be considered as an independent differential diagnostic tool for assessing the likelihood of an individual being afflicted by a particular brain-related disease, at the time of testing, or prospectively, with a certain probability predictive power, (e.g., in conjunction with the present invention's NEURODIAGNOSTICS MODULE 101, the REGIONS OF INTEREST COMPUTATIONAL MODULE 102, or as constituting an altogether independent differential diagnostic neurobehavioral tool).

More specifically, as the REGIONS OF INTEREST COMPUTATIONAL MODULE 102 may include any one of the three INDIVIDUAL FUNCTIONAL ACTIVATION DATA 116, INDIVIDUAL STRUCTURAL MAPS 118, or INDIVIDUAL COGNITIVE PROFILE in any possible combination or separately—together with the FUNCTIONAL STRUCTURAL COGNITIVE NORM DATA 121, the STANDARD BRAIN REGIONS DEVIATION ANALYSIS 122 is capable of outputting the REGIONS OF INTEREST DATA 124 as either the functional, structural, or cognitive statistically significant deviant features of an individual. Accordingly, the BRAIN TRAIT THRESHOLD COMPUTATION 126 is capable of differentially diagnosing the likelihood of an individual being afflicted with a particular brain-related disease based on functional, structural, or cognitive deviant REGIONS OF INTEREST DATA 124 (separately or together, in any possible combination).

As such, the BRAIN TRAIT COMPUTATION MODULE 103 is also capable of offering a differential diagnostic tool for assessing the likelihood of an individual either being afflicted with a particular brain disease, at the time of testing, or prospectively, within set periods of time based on the INDIVIDUAL FUNCTIONAL ACTIVATION DATA 116, INDIVIDUAL STRUCTURAL MAPS 118, or INDIVIDUAL COGNITIVE PROFILE 120 separately or in any combination. Hence, the BRAIN TRAIT COMPUTATION MODULE 103 may also function as a separate or independent neurobehavioral differential diagnostic tool that is capable of screening the wide population for any existent or prospective brain-related disease (or alternatively for enhanced cognitive performance capabilities in an individual) based on either a simple COGNITIVE DATA 112 (derived from various cognitive or behavioral testing) which is analyzed by the STATISTICAL COMPUTATION MODULE 114 and leads to the INDIVIDUAL COGNITIVE PROFILE 120, or based on more extensive FUNCTIONAL NEUROIMAGING 108 and STRUCTURAL NEUROIMAGING DATA 111 that are analyzed again by the STATISTICAL COMPUTATION MODULE 114 and lead to the INDIVIDUAL FUNCTIONAL ACTIVATION DATA 116 and INDIVIDUAL STRUCTURAL MAPS 118 and the above-mentioned COGNITIVE DATA 112 (in any possible combination).

Indeed, given the low-cost of a preliminary screening testing which obtains only COGNITIVE DATA 112 (which nevertheless can be computed by the STATISTICAL COMPUTATION MODULE 114 and STANDARD BRAIN REGIONS DEVIATION ANALYSIS 122 thereby yielding a statistically significant differential diagnostic or predictive diagnostic capabilities), such cognitive or behavioral testing may be used as an initial wide-population screening tool for the existence or likelihood for the development of various brain-related diseases. Following such low-cost generalized screening testing for the general population for a particular brain disease or diseases (which has a fair-to-good differential diagnostic, or prospective predictive diagnostic power), one could utilize a second-tier, more sophisticated, yet costly, full NEURODIAGNOSTICS MODULE 101 utilization of INDIVIDUAL FUNCTIONAL ACTIVATION DATA 116, INDIVIDUAL STRUCTURAL MAPS 118, and INDIVIDUAL COGNITIVE PROFILE 120 (or any combination thereof) to obtain a much more accurate (with a lower rate of false-positive) differential diagnosis of the particular brain disease.

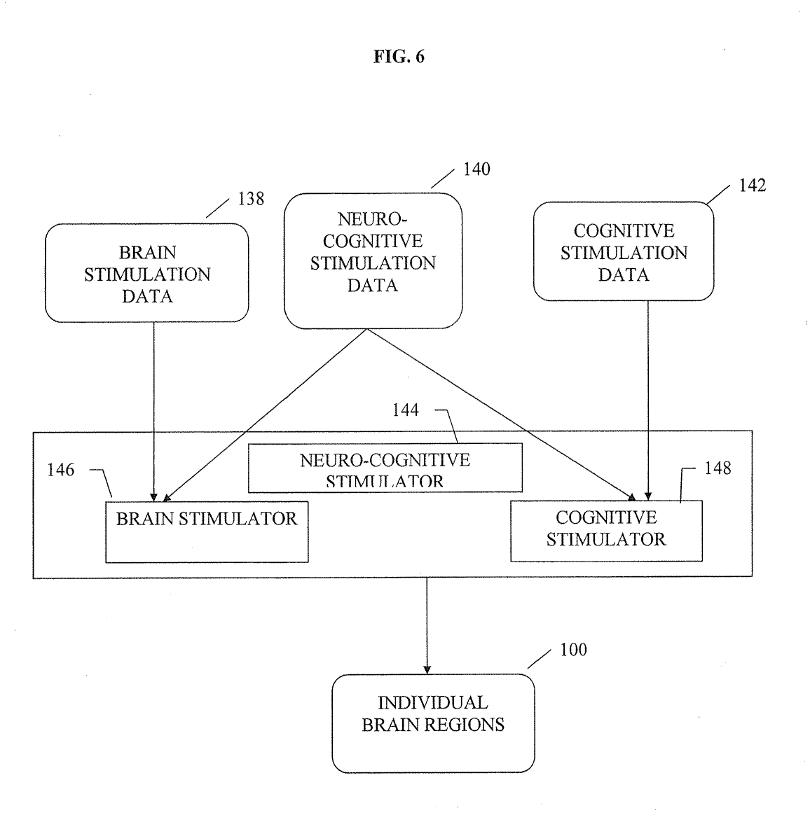

Reference is now made to FIG. 6 which details the STIMULATION MODULE 105 of the system 200 of FIG. 1. The STIMULATION MODULE 105 is configured to stimulate particular brain regions and their corresponding cognitive stimulation in a given individual. The STIMULATION MODULE 105 includes the BRAIN STIMULATION DATA 138, the COGNITIVE STIMULATION DATA 140, and a NEURO-COGNITIVE STIMULATION DATA 140 of FIG. 5, which are input into the NEURO-COGNITIVE STIMULATOR 144. In turn, the NEURO-COGNITIVE STIMULATOR 144 includes a BRAIN STIMULATOR 146 and a COGNITIVE STIMULATOR 148. Specifically, the BRAIN STIMULATION DATA 138 and the NEURO-COGNITIVE STIMULATION DATA 140 are input into the BRAIN STIMULATOR 146, and the NEURO-COGNITIVE STIMULATION DATA 140 and COGNITIVE STIMULATION DATA 142 are input into the COGNITIVE STIMULATOR 148. Based on the BRAIN STIMULATION DATA 138, the COGNITIVE STIMULATION DATA 140, and the NEURO-COGNITIVE STIMULATION DATA 140, the BRAIN STIMULATOR 146 and the COGNITIVE STIMULATOR 148 determine the INDIVIDUAL BRAIN REGIONS 100, which is the actual stimulation of the identified brain region or regions, and which includes an inhibitory or excitatory brain and cognitive stimulation according to particular stimulation parameters determined by the TRAIT-THRESHOLD INVERSE STIMULATION COMPUTATION 132.

An exemplary embodiment of the NEURO-COGNITIVE STIMULATOR 144 includes an integrated BRAIN STIMULATOR 146 and the COGNITIVE STIMULATOR 148, which can stimulate the same INDIVIDUAL BRAIN REGIONS 100 simultaneously or with time-separation between the brain loci and corresponding cognitive stimulation of these brain loci in any possible order. Thus, the NEURO-COGNITIVE STIMULATOR 144 stimulates single or multiple INDIVIDUAL BRAIN REGIONS 100 loci with excitatory or inhibitory brain stimulation parameters including the varying of: the intensity or duration or interval of each of the stimulation brain loci separately or together, while also varying the cognitive “excitatory” or “inhibitory” stimulation of each of these brain loci separately or together (e.g., providing cognitive stimulation or training for each of the stimulated brain regions which corresponds to the excitatory or inhibitory feature of the brain stimulation of a particular loci). For example, an excitatory 10-20 Hz TMS of the left prefrontal cortex aimed at improving or enhancing the mood or well-being of an individual can be coupled with a computerized, auditory, or visual presentation of a Beck-based “positive thinking,” or change in self-construct cognitive stimulation or training paradigm, which may be juxtaposed together in any possible order and with any temporal separation between their onset, termination time, and length of stimulation.

Likewise, an excitatory 10-20 Hz TMS stimulation of the cingulate gyrus geared towards improving concentration or focus, or in conjunction with temporal or hippocampal excitatory 10-20 Hz TMS stimulation to improve deficient memory, executive function, or concentration capabilities or enhance them, can be coupled with a juxtaposition in any temporal order and length or intensity of excitatory cognitive stimulation or training, which may consist of short term memory cognitive exercises or attention allocation exercises. Alternatively, an inhibitory 1 Hz TMS stimulation of diseased Schizophrenic right hemispheric temporal or parietal associated delusional “visions” or “sounds” may be coupled, in any order and temporal length or intensity, with a cognitive stimulation or training geared towards diminishing the likelihood of occurrence of false-perceptions (e.g., through enhanced perceptual training such as enhancing perceptual cues in perceptual illusion paradigms or other perceptual paradigms or, alternatively, through enhancing accurate perception training or through cognitive stimulation or training in enhancing attention or attentional allocation capabilities, or increasing psychophysical judgment capabilities). Alternatively, individuals who have been characterized as possessing functional, structural, or cognitive abnormalities that are characteristic of autism may be stimulated by the NEURO-COGNITIVE STIMULATOR 144 through a combination of excitatory 10-20 Hz TMS stimulation of the LH's Broca's and Wernicke's regions and an inhibitory 1 Hz TMS of the abnormally hyperactivated (or structurally enlarged) contralateral RH's Broca's and Wernickes' language regions, that are coupled with cognitive or behavioral stimulation geared towards enhancing language development, articulation, naming, pointing, or joint attention skills, among others.

In yet another exemplary embodiment, the NEURO-COGNITIVE STIMULATOR 144 can also facilitate neuroplasticity changes geared towards improving functional, structural, or corresponding cognitive performance capabilities associated with a particular brain disease or, alternatively, geared towards enhancing a particular cognitive function or functions through an excitatory or inhibitory brain stimulation of single or multiple INDIVIDUAL BRAIN REGIONS 100 brain loci, which is combined with “opposite direction” inhibitory or excitatory cognitive stimulation. An example of such “opposite-direction” brain stimulation and cognitive stimulation can be the inhibitory 1 Hz TMS brain stimulation of the Amygdala or fusiform gyrus (which have been shown to be hyperactivated in ASD individuals during facial recognition and social cognition tasks, or during non-social communication paradigms or even at resting conditions) during resting conditions or during the conductance of non-social cognition tasks—which may be coupled with focused social cognition stimulation exercises (before or after the inhibitive TMS stimulation during the resting state or non-social communication tasks). Alternatively, the NEURO-COGNITIVE STIMULATOR 144 may activate the BRAIN STIMULATOR 146 or COGNITIVE STIMULATOR 148 separately, or with opposite excitatory vs. inhibitory stimulation parameters, for the same or different brain loci at the same or different time points or intervals.

The NEURO-COGNITIVE STIMULATOR 144 is also capable of dynamically adjusting or altering the intensity or interval of brain or cognitive stimulation of single or multiple INDIVIDUAL BRAIN REGIONS 100 brain loci, or the temporal juxtaposition of single or multiple brain stimulation loci and their corresponding cognitive stimulation based on potential changes in the TRAIT-THRESHOLD INVERSE STIMULATION COMPUTATION 132 that can arise as a result of the post-stimulation feedback measurement by the NEURODIAGNOSTICS MODULE 101 and subsequent computations by the REGIONS OF INTEREST COMPUTATIONAL MODULE 102, the BRAIN TRAIT COMPUTATION MODULE 103, and the TREATMENT MODULE 105.

In yet another embodiment, the NEURO-COGNITIVE STIMULATOR 144, the BRAIN STIMULATOR 146 and the COGNITIVE STIMULATOR 144 form a single integrated medical device, which is capable of synchronizing the brain stimulation of single or multiple brain INDIVIDUAL BRAIN REGIONS 100 loci together with the cognitive stimulation of the same brain loci, which may be controlled by the TRAIT-THRESHOLD INVERSE STIMULATION COMPUTATION 132 output BRAIN STIMULATION DATA 138, the COGNITIVE STIMULATION DATA 140, and the NEURO-COGNITIVE STIMULATION DATA 140. Alternatively, the NEURO-COGNITIVE STIMULATOR 144 can consist of at least two separate medical devices of the BRAIN STIMULATOR 146 and the COGNITIVE STIMULATOR 148 that are controlled by the same TRAIT-THRESHOLD INVERSE STIMULATION COMPUTATION 132 through its output of the BRAIN STIMULATION DATA 138, the COGNITIVE STIMULATION DATA 140, and the NEURO-COGNITIVE STIMULATION DATA 140.

The COGNITIVE STIMULATOR 148 may be of single or multiple presentation of various sensory modality stimulation such as visual, auditory, and tactile, for example, with various response modalities being used in any possible combination, including but not limited to a keypress response, vocal, written, tactile, or visually guided response with or without a response feedback element (e.g., which provides a feedback as to the accuracy of the subject's response or performance at different time points, or with regards to various segments of the task or tasks at hand).

The BRAIN STIMULATOR 146 may include a medical device capable of stimulating electromagnetically, electrically, magnetically, and/or photoelectrically, and inhibitorily or excitatorily, a single or multiple INDIVIDUAL BRAIN REGIONS 100 brain pixels, regions, tissues, functional neural units, or hemispheres, which have been deemed as functionally, structurally, or cognitively diseased by the BRAIN TRAIT THRESHOLD COMPUTATION 126 and based on the control of the BRAIN STIMULATION ANALYZER 133 and the direct input of the BRAIN STIMULATION DATA 138. Alternatively, the BRAIN STIMULATOR 146 may be a medical device capable of stimulating electromagnetically, electrically, magnetically, or photoelectrically, a single or multiple brain pixels, regions, tissues, functional neural units or hemispheres, which are functionally or structurally associated with a particular sub-enhanced cognitive function or functions by the BRAIN TRAIT THRESHOLD COMPUTATION 126 and based on the control of the BRAIN STIMULATION ANALYZER 133 and the direct input of the BRAIN STIMULATION DATA 138.

In yet another embodiment, the BRAIN STIMULATOR 146 may include a medical device capable of stimulating electromagnetically, electrically, magnetically, and/or photoelectrically, and inhibitorily or excitatorily, a single or multiple brain pixels, regions, tissues, functional neural units, or hemispheres through the convergence of at least two electrical, magnetic, electromagnetic, or photoelectric sources of energy or stimulation, in any possible combination. These single or multiple electrical, magnetic, electromagnetic, or photoelectric sources can be placed at any point on top of the cranium or surface of the scalp, or face or neck, broadly defined or non-invasively within any of the orifices located in the head, e.g., the ears, nose, sinuses, mouth and larynx, eyes. Additionally, each of these stimulating or receiving electrical, magnetic, electromagnetic, or photoelectric sources is controlled individually or collectively by the NEURO-COGNITIVE STIMULATOR 144 and specifically through the dynamic input from the BRAIN STIMULATION DATA 138.

Following the ROI NEUROCOGNITIVE STIMULATION 150, feedback measurements are performed by the NEURODIAGNOSTICS MODULE 101, REGIONS OF INTEREST COMPUTATIONAL MODULE 102, BRAIN TRAIT COMPUTATION MODULE 103, TREATMENT MODULE 104, and STIMULATION MODULE 105, as depicted in FIG. 1, and as detailed above. The inclusion of such a “feedback loop” (i.e., from the STIMULATION MODULE 105 to the NEURODIAGNOSTICS MODULE 101) allows to monitor and adjust the individual disease-based or cognitive enhancement stimulation parameters continuously following stimulation. It also allows for a dynamic automatic learning taking place at the TREATMENT MODULE 104, i.e., in terms of the TRAIT-THRESHOLD INVERSE STIMULATION COMPUTATION 132 optimization—for a particular disease, or individual based on a comparison of the pre- and post-stimulation ROI THRESHOLD DATA 128 and BRAIN CONDITION DATA 129 (namely, a statistical meta-analysis or any other statistical procedure which is capable of cumulatively assessing the relationship between varying the pre-stimulation parameters output by the BRAIN STIMULATION ANALYZER 133, COGNITIVE STIMULATION ANALYZER 134, or NEURO-COGNITIVE STIMULATION ANALYZER 136 for a specific BRAIN CONDITION DATA 129 disease or particular cognitive enhancement protocol and particular ROI THRESHOLD DATA 128 and the post-stimulation measured ROI THRESHOLD DATA 128 and BRAIN CONDITION DATA 129, to determine the most effective brain stimulation, cognitive stimulation and corresponding neuro-cognitive stimulation parameters).

Although the present invention has been described in connection with preferred embodiments, many modifications and variations will become apparent to those skilled in the art. While preferred embodiments of the invention have been described and illustrated above, it should be understood that these are exemplary of the invention and are not to be considered as limiting. Accordingly, it is not intended that the present invention be limited to the illustrated embodiments, but only by the appended claims.

1. A device for assessing and enhancing particular cognitive, behavioral, or affective functions in a patient comprising:
a neurodiagnostic module for identifying an individual's brain regions of interest and associated with particular functional, structural, or cognitive abnormalities and measuring the functional activation or structural maps, or corresponding cognitive performance for a particular task (or tasks) or during a resting period;
a computational module identifying the particular brain regions in an individual whose structure, function, or cognitive functions are deviant from their corresponding statistically-established health norms, or from their corresponding statistical norms for cognitively enhanced performance in a particular task;
a brain trait module for determining whether the identified brain regions from said computational module statistically fits within known structural, functional, or cognitive pathophysiology of a particular brain-related disease, or within established norms for enhanced or excellent cognitive or behavioral performance for a particular task;
a treatment module for computing precise individual-based brain and cognitive stimulation parameters needed to stimulate the particular identified brain regions; and
a stimulator module system for distributing, at least non-invasively, energy to stimulate the identified brain regions associated with the particular functional, structural, or cognitive abnormalities.

2. The device of claim 1, wherein the stimulator module system comprises:
at least one of a brain stimulator and a cognitive stimulator, the brain stimulator being configured to selectively stimulate brain regions deviant from their corresponding statistically-established health norms, or from their corresponding statistical norms for cognitively enhanced performance in a particular task.

3. The device of claim 2, further including at least one electrode in communication with at least one of ear, nose, scalp and mouth of an individual.

4. The device of claim 2, wherein the brain stimulator comprises at least one invasive stimulator operative to selectively provide singular or multiple site stimuli to one or more identified brain regions.

5. The device of claim 1, wherein the brain stimulator generates an excitatory or inhibitory stimulus energy.

6. The device of claim 1, wherein the brain stimulator module is configured to provide at least one of electrical, magnetic, electromagnetic and photoelectric stimulus.

7. The device of claim 1, wherein the treatment module further includes ROI threshold data and brain condition data that inputs the data into a trait-threshold inverse stimulation computation.

8. The device of claim 8, wherein the treatment module further includes a system of analyzers.

9. The device of claim 8, wherein said system of analyzers includes a brain stimulation analyzer, a cognitive stimulation analyzer, and a neuro-cognitive stimulation analyzer.

10. The device of claim 1, wherein the stimulation module system includes brain stimulation data, cognitive stimulation data, neuro-cognitive stimulation data, and a correspondingly interfaced brain stimulator, cognitive stimulator and neuro-cognitive stimulator.

11. The device of claim 1, wherein the neurodiagnostic module is operative to identify brain regions associated Alzheimer's disease, dementia, autism spectrum disorder, mild cognitive impairment, memory loss, aging, ADHD, Parkinson's disease, depression, addiction, substance abuse, schizophrenia, bipolar disorder, memory enhancement, intelligence enhancement, concentration enhancement, well-being or mood enhancement, self-esteem enhancement, language capabilities, verbal skills, vocabulary skills, articulation skills, alertness, focus, relaxation, perceptual skills, thinking, analytical skills, executive functions, sleep enhancement, motor skills, coordination skills, sports skills, musical skills, inter-personal skills, social skills and affective skills.

12. The device of claim 1, wherein said stimulator module system is operative to at least non-invasively apply stimulus energy to a left prefrontal region, frontal lobes, cingulate gyrus, hemispheres, temporal lobe, frontal lobe, parietal lobe, occipital lobe, amygdala region, cerebellum, hippocampus, anthreonal, Peabody, plaques, tangles, brain stem, medula, corpus collasum, subcortical region, cortex, gyrus, white matter and grey matter.

13. The device of claim 1, wherein the neurodiagnostic module is operative to identify brain regions associated Alzheimer's disease and said stimulator module system is operative to at least non-invasively apply stimulus energy to an individual brain regions associated with Alzheimer's disease.

14. The device of claim 13, wherein the analyzer system is operative to measure at least one property of an individual's condition to obtain a measured property, the measured property being related to at least one of at least one brain region and at least one cognitive feature.

15. The device of claim 14, wherein the first stimulator comprises at least one non-invasive brain stimulator and at least one invasive brain stimulator, wherein the at least one non-invasive brain stimulator and the at least one invasive brain stimulator are configured to selectively stimulate the at least one brain region.

16. A system for assessing patient neuronal functions:
a neurodiagnostic module for identifying an individual's brain regions of interest and measuring the functional activation or structural maps, or corresponding cognitive performance thereof;
a computational module identifying a patients deviant brain regions relative to corresponding statistically-established health norms, or from their corresponding statistical norms;
a brain trait module for determining whether the identified brain regions from said computational module statistically fits within known structural, functional, or cognitive pathophysiology of a particular brain-related disease, or within established norms for enhanced or excellent cognitive or behavioral performance for a particular task; and
a treatment system computing precise individual-based brain and cognitive stimulation parameters needed to stimulate generating at least non-invasively excitatory or inhibitory energy to stimulate the identified brain regions.

17. A method of assessing and treating cognitive, behavioral, or affective functions in a patient comprising:
neurodiagnostically identifying an individual's brain regions of interest and associated with particular functional, structural, or cognitive abnormalities and measuring the functional activation or structural maps, or corresponding cognitive performance for a particular task (or tasks) or during a resting period;
identifying brain regions in an individual whose structure, function, or cognitive functions are deviant from their corresponding statistically-established health norms, or from their corresponding statistical norms for cognitively enhanced performance in a particular task;
determining whether the identified brain regions from said computational module statistically fits within known structural, functional, or cognitive pathophysiology of a particular brain-related disease, or within established norms for enhanced or excellent cognitive or behavioral performance for a particular task;
computing precise individual-based brain and cognitive stimulation parameters needed to stimulate the particular identified brain regions; and
at least non-invasively discharging energy to stimulate the identified brain regions associated with the particular functional, structural, or cognitive abnormalities. A system for assessing patient neuronal functions

18. A method of assessing and treating cognitive, behavioral, or affective functions in a patient comprising:
providing imaging devices to assist with identifying a patients deviant brain regions relative to corresponding statistically-established health norms, or from their corresponding statistical norms;
determining whether the identified brain regions statistically fits within known structural, functional, or cognitive pathophysiology of a particular brain-related disease, or within established norms for enhanced or excellent cognitive or behavioral performance for a particular task; and
a treatment system computing precise individual-based brain and cognitive stimulation parameters needed to stimulate generating at least non-invasively excitatory or inhibitory energy to stimulate the identified brain regions.

19. A method of treating cognitive impairments, comprising the steps of:
identifying at least one brain region associated with a cognitive impairment;
subjecting the at least one brain region to at least non-invasive excitatory or inhibitory stimulus energy; and
simultaneously, modifying a cognitive function associated with the at least one brain region.

20. The method of claim 19, further comprising the steps of:
subjecting the brain region to a treatment including at least one of cell replacement therapy, cell regenerative therapy and cell growth; and
optionally, subjecting the brain region to a pharmacological treatment.

21. A method of therapy for impaired cognitive functions, the method comprising the steps of:
providing a first stimulus to a pre-defined brain region of an individual without stimulating physically adjacent brain regions, the pre-defined brain region being functionally associated with an impaired cognitive function; and
providing at least one cognitive stimulus to the patient, to elicit a response involving the impaired cognitive function.

22. The method of claim 21, further comprising the steps of:
measuring at least one local brain function of the pre-defined brain region to obtain a local measurement;
comparing the local measurement to a normative value to obtain an evaluation data; and
adjusting, in response to the evaluation data, at least one of the steps of providing a first stimulus and providing at least one cognitive stimulus.

23. The method of claim 22, further comprising:
measuring the response to the first stimulus and to the cognitive stimulus, to obtain a cognition data; and
adjusting, based on the cognition data, at least one of the steps of providing a first stimulus and providing at least one cognitive stimulus.

24. A method of brain therapy, comprising:
providing an analyzer system including at least one of a brain analyzer and a cognitive analyzer;
the analyzer system measuring at least one property of an individual's condition to obtain a measured property related to at least one of at least one brain region and at least one cognitive feature;
the analyzer system calculating a norm value from at least one brain region or at least one cognitive feature of healthy or diseased groups of individuals; and
the analyzer system comparing the measured property value with the norm value to provide a resultant value.

25. The method of claim 24, further including providing a neuro-cognitive stimulation analyzer.

26. A method of therapy, comprising:
measuring a local brain function of at least one brain region to produce a local measurement relative to a norm value of an individual;
evaluating the local measurement relative to a normative value of a group of individuals, to produce an evaluation datum;
adjusting at least one of one or more non-invasive stimulators and one or more one cognitive stimulators in response to the evaluation datum; and
providing stimuli from the at least one or more non-invasive stimulators and the least one or more cognitive stimulators to the at least one brain region.

27. The method of claim 26, further comprising:
measuring a response to the stimuli to produce a cognition measurement; and
adjusting at least one of the non-invasive and cognitive stimulators in response to the cognition measurement.

28. The method of claim 26, wherein the step of providing non-invasive stimuli includes providing electromagnetic stimuli and another form of neuronal stimuli, to selectively stimulate the at least one brain region.

29. A method of therapy for a cognitive symptom of a disease, comprising:
selectively stimulating at least one brain region functionally associated with a cognitive symptom, without physically stimulating adjacent regions; and
providing at least one cognitive stimulus selected to elicit a response involving the cognitive symptom.

30. The method of claim 29, further comprising:
measuring local brain function at the at least one brain region to produce a local measurement;
evaluating the local measurement relative to a normative value to produce an evaluation datum; and
adjusting at least one of the brain region stimulating and the at least one cognitive stimulus in response to the evaluation datum.

31. The method of claim 30, further comprising:
measuring the response to the stimulus to produce a cognition measurement; and
adjusting at least one of the selective stimulating and the at least one cognitive stimulus in response to the cognition measurement.

32. The method of claim 31, wherein the at least one brain region is a brain deficient region.

33. The method of claim 32, wherein the at least one brain region is associated with a memory function disease.

34. The method of claim 32, wherein the at least one brain region is associated with Alzheimer's disease.

35. A method of neuronal therapy comprising:
providing an analyzer system including at least one of a brain analyzer and a cognitive analyzer;
the analyzer measuring at least one property of an individual's condition, the measured property being related to at least one of at least one brain region and at least one cognitive feature;
the analyzer calculating a norm value from at least one brain region or at least one cognitive feature of healthy or diseased groups of individuals;
the analyzer comparing the measured property with the norm value and provide a resultant value;
providing a stimulator system including:
providing one or more non-invasive stimulators to selectively provide singular or multiple site stimuli to at least one brain region associated with the property;
providing one or more cognitive stimulators to selectively provide cognitive stimuli to at least one cognitive function relevant to the at least one property; and
providing a controller and interfacing the controller with the analyzer system and the stimulator system, the controller operating at least one of the one or more non-invasive brain stimulators and one or more one cognitive stimulators.
